SEQUENCE LISTING (4 sequences)

SEQ ID NO: 1 - DNA, 20 bp, synthetic construct
  source 1..20: mol_type = other DNA
agcgcagacgggtcgttaag

SEQ ID NO: 2 - DNA, 23 bp, synthetic construct
  source 1..23: mol_type = other DNA
tcacatcagacttaaaagaccgc

SEQ ID NO: 3 - DNA, 20 bp, synthetic construct
  source 1..20: mol_type = other DNA
agcgcagacgggtcgttaag

SEQ ID NO: 4 - DNA, 20 bp, synthetic construct
  source 1..20: mol_type = other DNA
gtgtgctgccttcgcaatcg

(19) United States
(12) Patent Application Publication
(10) Pub. No.: US 2026/0297688 A1
(43) Pub. Date: Oct. 1, 2026

(54) BIOMARKER COMPRISING LACTOBACILLUS SALIVARIUS OR BACTEROIDES PLEBEIUS FOR PREDICTING THERAPEUTIC RESPONSIVENESS TO AND PROGNOSIS OF TREATMENT WITH AN IMMUNE ANTICANCER AGENT, AND METHOD FOR PREDICTING THERAPEUTIC RESPONSIVENESS TO AND PROGNOSIS OF TREATMENT WITH AN IMMUNE ANTICANCER AGENT USING SAME

(71) Applicants: THE ASAN FOUNDATION, Seoul (KR); UNIVERSITY OF ULSAN FOUNDATION FOR INDUSTRY COOPERATION, Ulsan (KR); GWANGJU INSTITUTE OF SCIENCE AND TECHNOLOGY, Gwangju (KR)

(72) Inventors: Sook Ryun PARK, Seoul (KR); Hansoo PARK, Seoul (KR); Yun Jae KIM, Jangseong-gun (KR)

(73) Assignees: THE ASAN FOUNDATION, Seoul (KR); UNIVERSITY OF ULSAN FOUNDATION FOR INDUSTRY COOPERATION, Ulsan (KR); GWANGJU INSTITUTE OF SCIENCE AND TECHNOLOGY, Gwangju (KR)

(21) Appl. No.: 19/489,011

(22) PCT Filed: May 31, 2024
(86) PCT No.: PCT/KR2024/007513

§ 371 Date: Dec. 1, 2025

(30) Foreign Application Priority Data
Jun. 1, 2023 (KR) 10-2023-0070783

(51) Int. Cl. C12Q 1/689; A61K 35/00; A61K 35/741; A61K 35/747; A61K 39/00; A61K 39/395; A61P 35/00

(52) U.S. Cl. CPC C12Q 1/689; A61K 35/741; A61K 35/747; A61K 39/3955; A61P 35/00; A61K 2035/115; A61K 2039/505; C12Q 2600/118

(57) ABSTRACT

A biomarker may include Lactobacillus salivarius or Bacteroides plebeius for predicting therapeutic responsiveness to and prognosis of treatment with an immune anticancer agent and a method may use such a biomarker. Lactobacillus salivarius and Bacteroides plebeius strains inhibit the therapeutic effect of immune anticancer agents in a cohort of patients treated with immune anticancer agents who have received fecal microbiota transplantation. Predicting therapeutic responsiveness to immune anticancer agent treatment or prognosis after immune anticancer agent treatment and securing the efficiency of existing fecal microbiota transplantation by detecting the strains. Since the selected strains are strains present in the human intestine and have excellent safety due to low therapeutic side effects, they may be advantageously used for the treatment of autoimmune diseases and the like through immune activity inhibition.

### TECHNICAL FIELD

The present invention relates to a biomarker comprising Lactobacillus salivarius or Bacteroides plebeius for predicting therapeutic responsiveness to and prognosis of treatment with an immune anticancer agent and a method for predicting therapeutic responsiveness to and prognosis of treatment with an immune anticancer agent using same.

This application claims priority to and the benefit of Korean Patent Application No. 10-2023-0070783, filed on Jun. 1, 2023, the disclosure of which is incorporated herein by reference in its entirety.

### BACKGROUND ART

Conventional cancer treatment is a method of removing as many cancer cells as possible from a patient through surgery, radiation therapy, and chemotherapy. However, surgical and radiation therapies are most effective for early-stage, non-metastatic cancers when cancer cells can be completely removed. While chemotherapy is widely used for cancer treatment, it has the disadvantage of low treatment efficacy for most solid tumors and is associated with various side effects. To improve such conventional problems, recently, immunotherapy using an immune anticancer agent is emerging.

Immune anticancer agents or cancer immunotherapy are treatments that activate the immune system of the human body to fight cancer cells, and in responsive patient groups, a high cure rate can be expected, thus offering a new paradigm in cancer therapy. However, the actual success rate of immune anticancer agents, such as anti-PD-1/anti-PD-L1 agents, is only 15 to 20%, indicating that the therapeutic responsiveness to immune anticancer agents is not high, and there are significant differences depending on cancer type and stage, and the condition of the patient. In addition, the activation of immune cells and secreted materials, which increase due to the use of immune anticancer agents, can also affect normal cells, leading to potential side effects.

Accordingly, considering the diversity in therapeutic responsiveness to cancer immunotherapy, active research is underway on companion diagnostics using biomarkers that can predict the therapeutic responsiveness to immune anticancer agents.

However, considering the high costs and low cure rate associated with cancer immunotherapy, there remains a need for biomarkers that can more accurately predict the therapeutic responsiveness to immune anticancer agents and for companion diagnostics using these biomarkers to provide personalized precise treatment to patients.

Therefore, the present inventors aimed to identify biomarkers that can predict responsiveness to or prognosis of cancer immunotherapy by confirming the gut microbiota in patients with high therapeutic responsiveness and low therapeutic responsiveness to immune anticancer agents.

### DISCLOSURE

### Technical Problem

As a result of confirming the microbial composition after fecal microbiota transplantation (FMT) in patients treated with an immune anticancer agent, the inventors of the present invention found that the Lactobacillus salivarius and Bacteroides plebeius strains were significantly decreased in patients showing improved therapeutic responsiveness to the immune anticancer agent after FMT, and thus completed the present invention based on these findings.

Accordingly, an object of the present invention is to provide a composition for predicting the therapeutic responsiveness to an immune anticancer agent, comprising an agent that detects one or more intestinal microorganisms selected from the group consisting of Lactobacillus salivarius and Bacteroides plebeius as an active ingredient, or a composition for predicting the prognosis of a cancer patient after treatment with an immune anticancer agent, comprising the same.

Another object of the present invention is to provide a kit for predicting the therapeutic responsiveness to an immune anticancer agent or a kit for predicting the prognosis of a cancer patient after treatment with an immune anticancer agent, comprising the composition and instructions.

Still another object of the present invention is to provide a method of providing information for predicting the therapeutic responsiveness to an immune anticancer agent or a method of providing information for predicting the prognosis of a cancer patient after treatment with an immune anticancer agent, comprising detecting one or more intestinal microorganisms selected from the group consisting of Lactobacillus salivarius and Bacteroides plebeius in a biological sample isolated from a subject.

Yet another object of the present invention is to provide a pharmaceutical composition for preventing or treating an autoimmune disease, comprising one or more intestinal microorganisms selected from the group consisting of Lactobacillus salivarius and Bacteroides plebeius as an active ingredient.

However, technical problems to be solved in the present invention are not limited to the above-described problems, and other problems which are not described herein will be fully understood by those of ordinary skill in the art from the following descriptions.

### Technical Solution

To achieve the above object, the present invention provides a composition for predicting the therapeutic responsiveness to an immune anticancer agent, comprising an agent that detects one or more intestinal microorganisms selected from the group consisting of Lactobacillus salivarius and Bacteroides plebeius as an active ingredient.

Additionally, the present invention provides a composition for predicting the prognosis of a cancer patient after treatment with an immune anticancer agent, comprising an agent that detects one or more intestinal microorganisms selected from the group consisting of Lactobacillus salivarius and Bacteroides plebeius as an active ingredient.

In one embodiment of the present invention, the immune anticancer agent may be one or more selected from the group consisting of an immune checkpoint inhibitor, an immune cell therapeutic agent, and an oncolytic viral therapeutic agent, but is not limited thereto.

In another embodiment of the present invention, the immune checkpoint inhibitor may be one or more selected from the group consisting of a PD-1 inhibitor, a PD-L1 inhibitor, a CTLA4 inhibitor, a LAG3 inhibitor, a TIM3 inhibitor, a 4-1BB inhibitor, a B7-H4 inhibitor, a VISTA inhibitor, a KIR inhibitor, and a BTLA inhibitor, but is not limited thereto.

In yet another embodiment of the present invention, the agent may be one or more selected from the group consisting of a primer pair, a probe, an antisense oligonucleotide, an antibody, and an aptamer, which specifically bind to 16S rRNA of the microorganism, but is not limited thereto.

In yet another embodiment of the present invention, the primer pair may comprise the base sequences of SEQ ID NOs: 1 and 2, or the base sequences of SEQ ID NOs: 3 and 4, but is not limited thereto.

In yet another embodiment of the present invention, the cancer may be one or more selected from the group consisting of colorectal cancer, rectal cancer, colon cancer, thyroid cancer, oral cancer, pharyngeal cancer, laryngeal cancer, cervical cancer, brain cancer, glioblastoma, lung cancer, ovarian cancer, bladder cancer, kidney cancer, liver cancer, pancreatic cancer, prostate cancer, skin cancer, tongue cancer, breast cancer, uterine cancer, stomach cancer, bone cancer, lymphoma, blood cancer, epithelial squamous cell cancer, adenocarcinoma of the lung, peritoneal cancer, skin cancer, cutaneous melanoma, ocular melanoma, anal cancer, esophageal cancer, small intestine cancer, endocrine cancer, parathyroid cancer, adrenal cancer, soft tissue sarcoma, urethral cancer, astrocytoma, endometrial cancer, salivary gland cancer, vulvar cancer, and head and neck cancer, but is not limited thereto.

Additionally, the present invention provides a kit for predicting the therapeutic responsiveness to an immune anticancer agent, comprising the composition for predicting the therapeutic responsiveness to an immune anticancer agent and instructions.

Additionally, the present invention provides a kit for predicting the prognosis of a cancer patient after treatment with an immune anticancer agent, comprising the composition for predicting the prognosis of a cancer patient after treatment with an immune anticancer agent and instructions.

Additionally, the present invention provides a method of providing information for predicting the therapeutic responsiveness to an immune anticancer agent, comprising detecting one or more intestinal microorganisms selected from the group consisting of Lactobacillus salivarius and Bacteroides plebeius in a biological sample isolated from a subject.

Additionally, the present invention provides a method of providing information for predicting the prognosis of a cancer patient after treatment with an immune anticancer agent, comprising detecting one or more intestinal microorganisms selected from the group consisting of Lactobacillus salivarius and Bacteroides plebeius in a biological sample isolated from a subject.

In one embodiment of the present invention, the biological sample may be one or more selected from the group consisting of blood, whole blood, plasma, stool, urine, tissue, cells, an organ, bone marrow, a fine-needle aspiration specimen, a fine-needle washout fluid, a core needle biopsy specimen, and saliva, but is not limited thereto.

In another embodiment of the present invention, the method of providing information for predicting the therapeutic responsiveness to an immune anticancer agent may further comprise predicting a low therapeutic responsiveness to an immune anticancer agent when the number of Lactobacillus salivarius or Bacteroides plebeius is higher than that of a control, but is not limited thereto.

In yet another embodiment of the present invention, the method of providing information for predicting the prognosis of a cancer patient after treatment with an immune anticancer agent may further comprise predicting a poor prognosis of a cancer patient after treatment with an immune anticancer agent when the number of Lactobacillus salivarius or Bacteroides plebeius is higher than that of a control, but is not limited thereto.

Additionally, the present invention provides a pharmaceutical composition for preventing or treating an autoimmune disease, comprising one or more intestinal microorganisms selected from the group consisting of Lactobacillus salivarius and Bacteroides plebeius or a culture fluid thereof as an active ingredient.

In one embodiment of the present invention, the autoimmune disease may be one or more selected from the group consisting of multiple sclerosis, inflammatory bowel disease, graft-versus-host disease, asthma, atopy, psoriasis, rheumatoid arthritis, and systemic lupus erythematosus, but is not limited thereto.

In another embodiment of the present invention, the composition may suppress immune activity, but is not limited thereto.

In yet another embodiment of the present invention, the composition may inhibit T cell proliferation or reduce interferon-gamma (IFN-γ) secretion of T cells, but is not limited thereto.

Additionally, the present invention provides a use of a composition comprising an agent that detects one or more intestinal microorganisms selected from the group consisting of Lactobacillus salivarius and Bacteroides plebeius as an active ingredient for predicting the therapeutic responsiveness to an immune anticancer agent.

Additionally, the present invention provides a use of an agent that detects one or more intestinal microorganisms selected from the group consisting of Lactobacillus salivarius and Bacteroides plebeius for preparing an agent for predicting the therapeutic responsiveness to an immune anticancer agent.

Additionally, the present invention provides a method of preventing or treating an autoimmune disease, comprising administering a composition that comprises one or more intestinal microorganisms selected from the group consisting of Lactobacillus salivarius and Bacteroides plebeius or a culture fluid thereof as an active ingredient to a subject in need thereof.

Additionally, the present invention provides a use of a composition comprising one or more intestinal microorganisms selected from the group consisting of Lactobacillus salivarius and Bacteroides plebeius or a culture fluid thereof as an active ingredient for preventing or treating an autoimmune disease.

Additionally, the present invention provides a use of one or more intestinal microorganisms selected from the group consisting of Lactobacillus salivarius and Bacteroides plebeius or a culture fluid thereof for preparing an agent for preventing or treating an autoimmune disease.

### Advantageous Effects

In the present invention, Lactobacillus salivarius and Bacteroides plebeius strains were selected as strains that inhibit the therapeutic effect of immune anticancer agents in a cohort of patients treated with immune anticancer agents who have received fecal microbiota transplantation (FMT). Accordingly, the present invention has the effect of predicting therapeutic responsiveness to immune anticancer agent treatment or prognosis after immune anticancer agent treatment and securing the efficiency of existing fecal microbiota transplantation by detecting the strains. In addition, since the selected strains are strains present in the human intestine and have excellent safety due to low therapeutic side effects, they are expected to be advantageously used for the treatment of autoimmune diseases and the like through immune activity inhibition.

### DESCRIPTION OF DRAWINGS

FIG. 1 is a schematic diagram illustrating the study design of a clinical trial according to one embodiment of the present invention.

FIG. 2 is a schematic diagram illustrating the combination therapy process of FMT and an anti-PD-(L)1 inhibitor according to one embodiment of the present invention.

FIG. 3 shows the results of confirming the microbial compositions of recipients R #1, R #2, and R #7 after FMT through beta-diversity analysis according to one embodiment of the present invention.

FIGS. 4A and 4B show the results of confirming the clinical efficacy for 13 FMT recipients according to one embodiment of the present invention.

FIGS. 4C and 4D show the results of confirming the α-fetoprotein (AFP) levels in FMT recipient R #7 after performing a first FMT using donor stool D #1 (FIG. 4C) or a second FMT using donor stool D #5 (FIG. 4D) according to one embodiment of the present invention.

FIG. 4E shows the results of confirming systemic immune changes in peripheral blood of FMT recipient R #7 according to one embodiment of the present invention.

FIG. 4F shows the results of confirming immune activation in the tumor microenvironment (TME) of FMT recipient R #7 according to one embodiment of the present invention.

FIG. 4G shows the examination results indicating the occurrence of immune-related gastritis due to immune activation after the second FMT in FMT recipient R #7 according to one embodiment of the present invention.

FIG. 5A shows the results of confirming changes in genus-level microbial abundance in FMT recipient R #7 according to one embodiment of the present invention.

FIG. 5B shows a Venn diagram illustrating the number of microorganisms in FMT recipient R #7 and the microorganisms that decreased after the second FMT compared to the first FMT according to one embodiment of the present invention.

FIG. 5C shows the results of confirming the abundance of the Bacteroides plebeius strain in FMT recipient R #7 after the first and second FMTs according to one embodiment of the present invention.

FIG. 5D shows the results of confirming the responsiveness to immune anticancer agent treatment in FMT recipients R #6 and R #7 according to one embodiment of the present invention.

FIG. 5E shows the results of comparing the abundance of B. plebeius between FMT recipients R #6 and R #7 according to one embodiment of the present invention.

FIG. 5F shows the results of confirming the abundance of the Lactobacillus salivarius strain in FMT recipients R #6 and R #7 after the first and second FMTs according to one embodiment of the present invention.

FIG. 6A shows the results of confirming the inhibitory effect of B. plebeius and L. salivarius strains on T cell proliferation according to one embodiment of the present invention.

FIG. 6B shows the results of confirming the inhibitory effect of B. plebeius and L. salivarius strains on interferon-gamma secretion of T cells according to one embodiment of the present invention.

FIG. 7 shows the results of confirming survival rates according to the presence or absence of B. plebeius and L. salivarius strains in an independent biomarker cohort according to one embodiment of the present invention.

### BEST MODE

In one experimental example of the present invention, as a result of confirming the microbial composition after fecal microbiota transplantation (FMT), it was confirmed that recipients R #1, R #2, and R #7 exhibited microbial compositions similar to those of their donors (see Experimental Example 1). Among them, as a result of confirming various clinical responses to immune anticancer agent administration in recipient R #7, who showed a partial response, it was found that there was no improvement in therapeutic responsiveness after the first FMT using donor stool D #1, whereas therapeutic responsiveness to the immune anticancer agent was improved after the second FMT using donor stool D #5 (see Experimental Example 2).

In another experimental example of the present invention, as a result of identifying the causative bacteria responsible for the therapeutic effect of FMT, the Bacteroides plebeius strain was found to be more abundant in the first FMT donor than in the second FMT donor for recipient R #7 and was significantly more abundant in R #7 after the first FMT than after the second FMT. Accordingly, it was confirmed that the B. plebeius strain is a strain that reduces the efficacy of an immune anticancer agent. In addition, as a result of analyzing the strains abundant in patient R #6, who did not show an improved therapeutic responsiveness after the second FMT compared to patient R #7, the Lactobacillus salivarius strain was selected. This strain was originally present in patient R #6, persisted or increased even after FMT, and was also present during the first FMT in patient R #7, who did not show an enhanced therapeutic responsiveness to the immune anticancer agent. Therefore, it was confirmed that this strain is a strain that reduces the efficacy of immune anticancer agents (see Experimental Example 3).

In yet another experimental example of the present invention, as a result of confirming the immunosuppressive activity of the B. plebeius and L. salivarius strains, it was confirmed that when culture fluids of the B. plebeius and L. salivarius strains were treated, the proliferation of both CD4 T cells and CD8 T cells was inhibited, and that the IFN-γ concentration was significantly reduced compared to the control when the culture fluids of the B. plebeius and L. salivarius strains were treated, thereby confirming that the B. plebeius and L. salivarius strains may suppress host immune activity (see Experimental Example 4).

In yet another experimental example of the present invention, as a result of confirming survival rates according to the presence or absence of the B. plebeius and L. salivarius strains, it was confirmed that the overall survival (OS) period was significantly longer in the patient group without the B. plebeius and L. salivarius strains (see Experimental Example 5).

Hereinafter, the present invention will be described in detail.

The present invention provides a composition for predicting the therapeutic responsiveness to an immune anticancer agent, comprising an agent that detects one or more intestinal microorganisms selected from the group consisting of Lactobacillus salivarius and Bacteroides plebeius as an active ingredient.

Additionally, the present invention provides a composition for predicting the prognosis of a cancer patient after treatment with an immune anticancer agent, comprising an agent that detects one or more intestinal microorganisms selected from the group consisting of Lactobacillus salivarius and Bacteroides plebeius as an active ingredient.

In the present invention, the microorganism Lactobacillus salivarius is not limited to a specific strain, as long as it is a bacterium known in the art or determined (classified) as a Lactobacillus salivarius species by a genome analysis method known in the art.

In the present invention, the microorganism Bacteroides plebeius is not limited to a specific strain, as long as it is a bacterium known in the art or determined (classified) as a Bacteroides plebeius species by a genome analysis method known in the art.

In the present invention, the agent may be one or more selected from the group consisting of a primer pair, a probe, an antisense oligonucleotide, an antibody, and an aptamer, which specifically bind to 16S rRNA of the microorganism, but is not limited thereto.

In the present invention, the term “primer” refers to a short nucleotide sequence having a free 3′ hydroxyl group that can form base pairs with a complementary template and functions as a starting point for template strand replication. In the present invention, a sense primer and an antisense primer may be provided as a pair (set). The primer may initiate DNA synthesis in the presence of reagents for a polymerization reaction (i.e., DNA polymerase or reverse transcriptase) and four different nucleoside triphosphates under appropriate buffer conditions and temperature. In the present invention, the therapeutic responsiveness to an immune anticancer agent may be determined (predicted) by performing PCR amplification and identifying whether the desired product is generated and/or whether the amount of the product increases. The PCR conditions and the lengths of the sense and antisense primers may be modified based on those known in the art.

In the present invention, “16S rRNA” is an rRNA constituting the 30S subunit of prokaryotic ribosomes, and since it contains conserved regions common to all species and hypervariable regions that can classify specific species, microorganisms may be identified through sequence analysis. In particular, since there is little diversity among individuals of the same species but diversity appears among different species, prokaryotes may be effectively identified by comparing the sequences of 16S IRNA. In addition, since 16S rDNA is a gene encoding 16S rRNA, microorganisms may also be identified using 16S rDNA.

In the present invention, the 16S rRNA of Lactobacillus salivarius may consist of or comprise the sequence of NCBI Reference Sequence: NR_028725.2 (https://www.ncbi.nlm.nih.gov/nuccore/NR_028725.2), but is not limited thereto.

In the present invention, the 16S rRNA of Bacteroides plebeius may consist of or comprise the sequence of GenBank: AB200217.1 (https://www.ncbi.nlm.nih.gov/nuccore/AB200217), but is not limited thereto.

In the present invention, the primer pair refers to a pair of primers that specifically bind to the 16S rRNA nucleotide sequences specifically present in Lactobacillus salivarius and Bacteroides plebeius.

In the present invention, the primer pair for Lactobacillus salivarius may consist of or comprise 5′-AGCGCAGACGGGTCGTTAAG-3′ (forward, SEQ ID NO: 1) and 5′-TCACATCAGACTTAAAAGACCGC-3′ (reverse, SEQ ID NO: 2), but is not limited thereto. The primer pair for Bacteroides plebeius may consist of or comprise 5′-AGCGCAGACGGGTCGTTAAG-3′ (forward, SEQ ID NO: 3) and 5′-GTGTGCTGCCTTCGCAATCG-3′ (reverse, SEQ ID NO: 4), but is not limited thereto.

In the present invention, the term “probe” refers to an oligonucleotide that has a sequence of a target nucleic acid, forms a double-stranded structure through complementary base pairing, and is typically labeled. The probe preferably includes a “hybridization region” corresponding to a region of the target sequence, which comprises at least 30 nucleotides and, in some cases, 50 or more nucleotides. The term “corresponding” means being identical or complementary to a specified nucleic acid. The probe preferably may not contain a sequence complementary to the sequence(s) used in preparing PCR. Generally, the 3′ end of the probe is “blocked” to inhibit incorporation of the probe into the primer extension product. The “blocking” may be achieved by using a non-complementary base or by adding a chemical moiety such as biotin, phosphate, or fluorophore to the 3′ hydroxyl group of the terminal nucleotide, which may serve both to block and to act as a label for subsequent detection or capture of the labeled nucleic acid, depending on the selected moiety. The blocking may be achieved by removing the 3′-OH group or by using a nucleotide lacking a 3′-OH group, such as a dideoxynucleotide.

In the present invention, the term “antisense oligonucleotide” is defined as an oligonucleotide capable of regulating the expression of a target gene by hybridizing to a target nucleic acid, particularly to a contiguous sequence on the target nucleic acid. The present invention includes modified or substituted oligomers comprising non-naturally occurring monomers or portions thereof that function similarly. Incorporation of substituted oligomers is based on factors such as enhanced cellular uptake or increased nuclease resistance and may be selected as known in the art. The entire oligonucleotide or a portion thereof may contain substituted oligomers. In addition, the antisense oligonucleotides of the present invention may include modified oligonucleotide mimetics, such as peptide nucleic acids (PNA) and locked nucleic acids (LNA), which are modified by methods known in the art to increase affinity for their targets and to provide tolerance to mismatches in the target sequence. Furthermore, the antisense oligonucleotides may be in the form of modified oligonucleotides including natural-type oligonucleotides, phosphorothioate oligo-deoxyribonucleotides, phosphorodithioate oligo-deoxyribonucleotides, methylphosphonate oligo-deoxyribonucleotides, phosphoramidate oligo-deoxyribonucleotides, H-phosphonate oligo-deoxyribonucleotides, triester oligo-deoxyribonucleotides, alpha-anomer oligo-deoxyribonucleotides, peptide nucleic acids, other synthetic nucleic acids, and nucleic acid-modified compounds, but are not limited thereto.

The length of the antisense oligonucleotide is not particularly limited, but is preferably 6 to 100 bases, more preferably 8 to 60 bases, and most preferably 10 to 40 bases. The antisense oligonucleotide may be synthesized in vivo or synthesized in vitro and administered in vivo according to conventional methods used in the art. A non-limiting example of a method for synthesizing antisense RNA in vivo includes using a vector in which the origin of the multiple cloning site (MCS) is in the opposite direction, so that the antisense RNA is transcribed. A non-limiting example of a method for synthesizing antisense RNA in vitro includes a method using RNA polymerase I.

In the present invention, the term “antibody specific to a microorganism” or “antibody against a microorganism” refers to a specific protein molecule that is directed against an antigenic site (or a marker protein distinguishing it from other entities) of a target microorganism (bacterium). For the purposes of the present invention, the antibody refers to an antibody that specifically binds to an antigenic site (or antigenic substance) of a microorganism (bacterium) and includes polyclonal antibodies, monoclonal antibodies, and recombinant antibodies. For the purposes of the present invention, it may be preferable that the antibody is a monoclonal antibody, which is a population of antibodies having substantially identical amino acid sequences in the heavy and light chains.

The production of antibodies as described above may be easily carried out using techniques well known in the art. Polyclonal antibodies may be produced by methods well known in the art, which involve injecting an antigen into an animal, collecting blood from the animal, and obtaining serum containing the antibodies. Such polyclonal antibodies may be prepared from any animal host species, including goats, rabbits, sheep, monkeys, horses, pigs, cattle, and dogs.

In the present invention, the term “aptamer” refers to a single-stranded DNA (ssDNA) or RNA having high specificity and affinity for a specific substance. The aptamer has extremely high affinity and stability for a specific substance, can be synthesized by a relatively simple method, allows various modifications to increase binding strength, and can target cells, proteins, and even small organic molecules, thereby exhibiting much higher specificity and stability compared to existing antibodies. Without being limited thereto, for example, the aptamer may be a small nucleic acid having a defined secondary and tertiary structure, such as a stem-loop structure, with a length of 15 to 50 bases, as known in the art. The aptamer may bind to a target molecule with very high specificity. In addition, the aptamer may be composed of a plurality of ribonucleotide units, deoxyribonucleotide units, or a mixture of the two types of nucleotide residues. The aptamer may further include one or more modified bases, sugars, or phosphate backbone units.

In the present invention, the term “immune anticancer agent” refers to a drug that enhances the body's inherent immune system to increase resistance against cancer. An immune anticancer agent has the advantage of fewer side effects because it treats through the patient's own immune enhancement, thereby improving the quality of life of cancer patients and significantly extending their survival time. The immune anticancer agent exerts its anticancer effect by enhancing the specificity, memory, and adaptiveness of the immune system. The immune anticancer agent includes, for example, an agent for immune checkpoint blockade (ICB), an immune cell therapeutic agent, an oncolytic viral therapeutic agent, a therapeutic antibody, or an immune checkpoint enhancer, but is not limited thereto. In the present invention, the agent for immune checkpoint blockade, that is, the immune checkpoint inhibitor, unlike conventional immunotherapeutic agents (such as cytokine therapies or cancer vaccines), binds to the binding site between cancer cells and T cells to block immune evasion signals, thereby preventing the formation of immunological synapses and enabling T cells, which are not subjected to immune evasion interference, to destroy cancer cells.

In the present invention, the immune checkpoint inhibitor may be one or more selected from the group consisting of a PD-1 inhibitor, a PD-L1 inhibitor, a CTLA4 inhibitor, a LAG3 inhibitor, a TIM3 inhibitor, a 4-1BB inhibitor, a B7-H4 inhibitor, a VISTA inhibitor, a KIR inhibitor, and a BTLA inhibitor, and may include, for example, nivolumab, atezolizumab, pembrolizumab, durvalumab, avelumab, ipilimumab, or tremelimumab, but is not limited thereto.

In the present invention, the term “detection” refers to both measuring and confirming the presence (expression) of a target substance, or measuring and confirming changes in the level (expression level) of the presence of the target substance. In the same context, detecting the microorganism in the present invention means measuring whether the microorganism is present (i.e., determining the presence or absence) or measuring qualitative or quantitative changes in the microorganism. The measurement may be carried out without limitation using both qualitative and quantitative methods (analyses). The types of qualitative and quantitative methods for measuring the presence of microorganisms are well known in the art, and the experimental methods described in the present specification are included therein.

In the present invention, the cancer may be one or more selected from the group consisting of colorectal cancer, rectal cancer, colon cancer, thyroid cancer, oral cancer, pharyngeal cancer, laryngeal cancer, cervical cancer, brain cancer, glioblastoma, lung cancer, ovarian cancer, bladder cancer, kidney cancer, liver cancer, pancreatic cancer, prostate cancer, skin cancer, tongue cancer, breast cancer, uterine cancer, gastric cancer, bone cancer, lymphoma, blood cancer, squamous cell carcinoma, lung adenocarcinoma, peritoneal cancer, skin cancer, cutaneous melanoma, ocular melanoma, anal cancer, esophageal cancer, small intestine cancer, endocrine gland cancer, parathyroid cancer, adrenal cancer, soft tissue sarcoma, urethral cancer, astrocytoma, endometrial cancer, salivary gland cancer, vulvar cancer, and head and neck cancer, but is not limited thereto.

Additionally, the present invention provides a kit for predicting the therapeutic responsiveness to an immune anticancer agent, comprising the composition for predicting the therapeutic responsiveness to an immune anticancer agent and instructions.

Additionally, the present invention provides a kit for predicting the prognosis of a cancer patient after treatment with an immune anticancer agent, comprising the composition for predicting the prognosis of a cancer patient after treatment with an immune anticancer agent and instructions.

In the present invention, the term “kit” refers to a tool that enables the prediction of therapeutic responsiveness to an immune anticancer agent or the prediction of prognosis of a cancer patient after treatment with an immune anticancer agent by including an agent for detecting the presence level of the microorganism. The kit of the present invention may further include, in addition to the above agent, other components, compositions, solutions, or devices that are conventionally required for the measurement or detection methods thereof. In this case, the material for measuring the presence level of the microorganism may be applied one or more times without limitation on the number of applications, there is no limitation on the order in which each material is applied, and the application of each material may be performed simultaneously or sequentially.

In the present invention, the kit may include a container, instructions, and an agent for detecting the presence level of the microorganism. The container may serve to package the agent and may also function to store and secure it. The material of the container may take the form of, for example, a bottle, tub, sachet, envelope, tube, or ampoule, and may be formed partially or entirely from plastic, glass, paper, foil, wax, or the like. The container may be equipped with a cap that is initially a part of the container or is completely or partially detachable and attachable to the container by mechanical, adhesive, or other means, and may also be equipped with a stopper that allows access to the contents with a syringe needle. The kit may include an external package, and the external package may include instructions for use of the components.

Additionally, the present invention provides a use of a composition comprising an agent that detects one or more intestinal microorganisms selected from the group consisting of Lactobacillus salivarius and Bacteroides plebeius as an active ingredient for predicting the therapeutic responsiveness to an immune anticancer agent.

Additionally, the present invention provides a use of an agent that detects one or more intestinal microorganisms selected from the group consisting of Lactobacillus salivarius and Bacteroides plebeius for preparing an agent for predicting the therapeutic responsiveness to an immune anticancer agent.

Additionally, the present invention provides a method of providing information for predicting the therapeutic responsiveness to an immune anticancer agent, comprising detecting one or more intestinal microorganisms selected from the group consisting of Lactobacillus salivarius and Bacteroides plebeius in a biological sample isolated from a subject.

Additionally, the present invention provides a method for predicting the therapeutic responsiveness to an immune anticancer agent, comprising detecting one or more intestinal microorganisms selected from the group consisting of Lactobacillus salivarius and Bacteroides plebeius in a biological sample isolated from a subject.

Additionally, the present invention provides a method of providing information for predicting the prognosis of a cancer patient after treatment with an immune anticancer agent, comprising detecting one or more intestinal microorganisms selected from the group consisting of Lactobacillus salivarius and Bacteroides plebeius in a biological sample isolated from a subject.

Additionally, the present invention provides a method for predicting the prognosis of a cancer patient after treatment with an immune anticancer agent, comprising detecting one or more intestinal microorganisms selected from the group consisting of Lactobacillus salivarius and Bacteroides plebeius in a biological sample isolated from a subject.

In the present invention, the intestinal microorganism may be measured by determining the amount of each microorganism-specific genome or gene using a primer set or probe that specifically binds to the 16S rRNA gene of the microorganism, but is not limited thereto. At this time, the amount of the microorganism-specific genome or gene may be measured using one or more methods selected from the group consisting of reverse transcription polymerase chain reaction (RT-PCR), competitive RT-PCR, real-time RT-PCR, quantitative or semi-quantitative RT-PCR, quantitative or semi-quantitative real-time RT-PCR, digital PCR, droplet digital PCR, in situ hybridization, fluorescent in situ hybridization (FISH), RNase protection assay (RPA), northern blotting, southern blotting, RNA sequencing, DNA chip, and RNA chip, but is not limited thereto.

In the present invention, the term “subject” may include both a cancer patient who is to receive treatment with an immune anticancer agent and a cancer patient who has received treatment with an immune anticancer agent.

In the present invention, the biological sample may be one or more selected from the group consisting of blood, whole blood, plasma, feces, urine, tissue, cells, organs, bone marrow, fine needle aspiration samples, fine needle washings, core needle biopsy samples, and saliva, and according to one embodiment or experimental example of the present invention, it may be feces, but is not limited thereto.

In the present invention, the method of providing information for predicting the therapeutic responsiveness to an immune anticancer agent or the method for predicting the therapeutic responsiveness to an immune anticancer agent may further comprise predicting a low therapeutic responsiveness to the immune anticancer agent when the number of Lactobacillus salivarius or Bacteroides plebeius is higher than that of a control group, but is not limited thereto. At this time, the control group may be cancer patients having a high therapeutic responsiveness to an immune anticancer agent, such as those showing a complete response (CR) or partial response (PR) after immune anticancer agent treatment, but is not limited thereto.

In the present invention, the method of providing information for predicting the therapeutic responsiveness to an immune anticancer agent or the method for predicting the therapeutic responsiveness to an immune anticancer agent may further comprise predicting a high therapeutic responsiveness to the immune anticancer agent when the number of Lactobacillus salivarius or Bacteroides plebeius is lower than or not detected compared to a control group, but is not limited thereto. At this time, the control group may be cancer patients having a low therapeutic responsiveness to an immune anticancer agent, such as those showing progressive disease (PD) or stable disease (SD) after immune anticancer agent treatment, but is not limited thereto.

In the present invention, the method of providing information for predicting the prognosis of a cancer patient after treatment with an immune anticancer agent, or the method for predicting the prognosis of a cancer patient after treatment with an immune anticancer agent, may further comprise predicting a poor prognosis of a cancer patient after treatment with an immune anticancer agent when the number of Lactobacillus salivarius or Bacteroides plebeius is higher than that of a control group, but is not limited thereto. At this time, the control group may be cancer patients having a good prognosis after immune anticancer agent treatment, such as those showing a complete response (CR) or partial response (PR) after immune anticancer agent treatment, but is not limited thereto.

In the present invention, the method of providing information for predicting the prognosis of a cancer patient after treatment with an immune anticancer agent, or the method for predicting the prognosis of a cancer patient after treatment with an immune anticancer agent, may further comprise predicting a good prognosis of a cancer patient after treatment with an immune anticancer agent when the number of Lactobacillus salivarius or Bacteroides plebeius is lower than or not detected compared to a control group, but is not limited thereto. At this time, the control group may be cancer patients having a poor prognosis after immune anticancer agent treatment, such as those showing progressive disease (PD) or stable disease (SD) after immune anticancer agent treatment, but is not limited thereto.

In the present invention, the term “prognosis prediction” refers to predicting the future tumor response or course of cancer after treatment with an immune anticancer agent in a cancer patient, and means predicting factors such as the probability of metastasis, recurrence, or metastatic relapse through the level of the microorganism.

Additionally, the present invention provides a method for providing information for determining or analyzing whether a subject has high sensitivity in predicting therapeutic responsiveness to an immune anticancer agent or prognosis after treatment with an immune anticancer agent, wherein after measuring the number of one or more microorganisms selected from the group consisting of Lactobacillus salivarius and Bacteroides plebeius in biological samples isolated from a subject suspected to have low therapeutic responsiveness to an immune anticancer agent or poor prognosis after treatment with an immune anticancer agent and from a control, if the difference in these levels increases (by twofold or more), it is determined to indicate that the subject has low therapeutic responsiveness to an immune anticancer agent or poor prognosis after treatment with an immune anticancer agent.

Additionally, the present invention provides a method for enhancing therapeutic responsiveness to an immune anticancer agent or increasing immune activity, comprising: detecting one or more intestinal microorganisms selected from the group consisting of Lactobacillus salivarius and Bacteroides plebeius in a biological sample isolated from a subject; and, when the number of Lactobacillus salivarius or Bacteroides plebeius is higher than that in a control group, performing fecal microbiota transplantation (FMT) from the control group to the subject.

In the present invention, the number of fecal microbiota transplantations (FMTs) is not limited, and, for example, may be 1 to 4 times, 1 to 3 times, or 1 to 2 times. The step of performing fecal microbiota transplantation from the control group may be a step of reducing the number of Lactobacillus salivarius or Bacteroides plebeius.

In the present invention, the control group in the method for enhancing therapeutic responsiveness to an immune anticancer agent or increasing immune activity may be cancer patients having high therapeutic responsiveness to an immune anticancer agent or cancer patients having a good prognosis after treatment with an immune anticancer agent, such as those showing a complete response (CR) or partial response (PR) after treatment with an immune anticancer agent, but is not limited thereto.

Additionally, the present invention provides a pharmaceutical composition for preventing or treating autoimmune diseases, comprising one or more intestinal microorganisms selected from the group consisting of Lactobacillus salivarius and Bacteroides plebeius, or a culture medium thereof, as an active ingredient.

In the present invention, Lactobacillus salivarius and Bacteroides plebeius are microorganisms present in the human intestine and may be isolated from human fecal samples, but are not limited thereto, and the origin thereof is not limited as long as they are bacteria known in the art or determined (classified) as Lactobacillus salivarius species and Bacteroides plebeius species by genome analysis methods known in the art.

In the present invention, the autoimmune disease is a disease in which the immune system of the human body attacks its own normal cells rather than external antigens, and may be one or more selected from the group consisting of multiple sclerosis, inflammatory bowel disease, graft-versus-host disease, asthma, atopy, psoriasis, rheumatoid arthritis, and systemic lupus erythematosus, but is not limited thereto.

In the present invention, the composition may suppress immune activity, but is not limited thereto.

In the present invention, the composition may inhibit T cell proliferation or decrease interferon-gamma (IFN-γ) secretion of T cells, but is not limited thereto.

Additionally, the present invention provides a method for preventing or treating an autoimmune disease, comprising administering to a subject in need thereof a composition comprising one or more intestinal microorganisms selected from the group consisting of Lactobacillus salivarius and Bacteroides plebeius, or a culture medium thereof, as an active ingredient.

Additionally, the present invention provides the use of a composition comprising one or more intestinal microorganisms selected from the group consisting of Lactobacillus salivarius and Bacteroides plebeius, or a culture medium thereof, as an active ingredient, for preventing or treating an autoimmune disease.

Additionally, the present invention provides the use of one or more intestinal microorganisms selected from the group consisting of Lactobacillus salivarius and Bacteroides plebeius, or a culture medium thereof for preparing an agent for preventing or treating an autoimmune disease.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, witepsol, polyethylene glycol, glycerogelatin, methylcellulose, carboxymethylcellulose, a mixture of stearic acid and oleic acid, subanal, cottonseed oil, peanut oil, palm oil, cacao butter+cholesterol, lecithin, lanette wax, glycerol monostearate, Tween or span, imhausen, monolan (propylene glycol monostearate), glycerin, adeps solidus, buytyrum tego-G, cebes pharma 16, hexalide base 95, cotomar, hydrokote SP, S-70-XXA, S-70-XX75 (S-70-XX95), hydrokote 25, hydrokote 711, idropostal, massa estrarium (A, AS, B, C, D, E, I, T), masa-MF, masupol, masupol-15, neosuppostal-N, paramount-B, supposiro (OSI, OSIX, A, B, C, D, H, L), suppository base IV types (AB, B, A, BC, BBG, E, BGF, C, D, 299), suppostal (N, Es), Wecobee (W, R, S, M, Fs), and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As used herein, the “subject” refers to a subject in need of treatment of a disease, and more specifically, refers to a mammal such as a human or a non-human primate, a mouse, a rat, a dog, a cat, a horse, and a cow.

As used herein, the “administration” refers to providing a subject with a predetermined composition of the present invention by using an arbitrary appropriate method. Accordingly, in the present invention, the term “administration” encompasses not only injecting or orally administering a substance to a subject, but also applying it to the subject.

The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention.

Hereinafter, preferred examples and experimental examples are presented to help understand the present invention. However, the following examples and experimental examples are provided only to help understand the present invention more easily, and the contents of the present invention are not limited by the following examples and experimental examples.

### EXAMPLES

### Example 1. Clinical Trial Design for Prospective Study

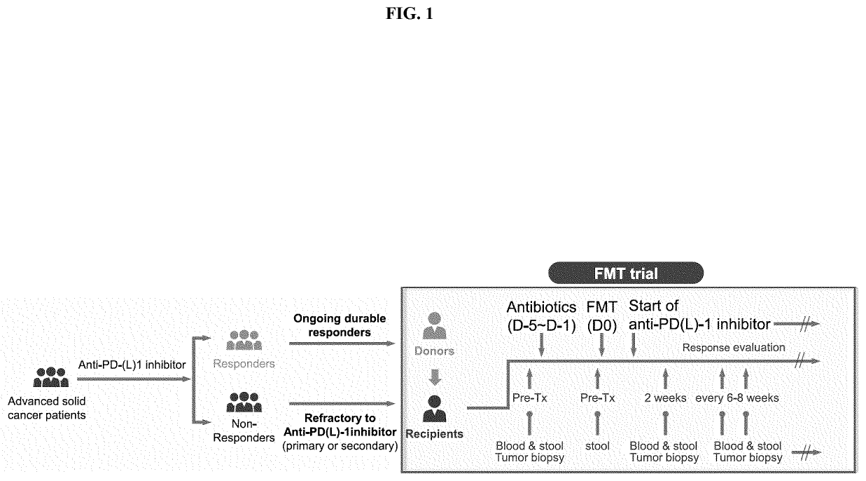

In the present invention, a prospective, single-arm, single-center clinical trial (NCT04264975) was conducted combining fecal microbiota transplantation (FMT) with anti-PD-(L)1 inhibitors in patients with advanced solid tumors refractory to an anti-PD-(L)1 inhibitor as an immune anticancer agent. The study design for this clinical trial is schematically shown in FIG. 1.

### Example 2. Selection of Donors and Recipients for FMT

Donors for FMT were selected based on the following criteria:
Age≥19
having histologically confirmed solid tumors (in the case of hepatocellular carcinoma, clinically confirmed diagnosis according to the American Association for the Study of Liver Diseases (AASLD) was also permitted)
sustained complete or partial response for at least 6 months to anti-PD-(L)1 monotherapy for unresectable or metastatic solid tumors according to RECIST v1.1
no concurrent infectious disease
no history of infectious diseases such as human immunodeficiency virus or other viral infections, or no risk behavioral characteristics, and no travel history to countries where endemic diarrhea occurs or a high risk of traveler's diarrhea within the past 6 months
no history of chronic gastrointestinal diseases including inflammatory bowel disease
no recent intake of allergens to which the recipient is known to be allergic
only donors who have passed serological tests and stool screening tests are considered suitable to donate stool samples for FMT

In addition, recipients of FMT were selected based on the following criteria:
Age≥19
Eastern Cooperative Oncology Group (ECOG) performance scale: 0-2
having histologically confirmed solid tumors (in the case of hepatocellular carcinoma, clinically confirmed diagnosis according to AASLD was also permitted)
disease progression was confirmed during anti-PD-(L)1-based treatment (monotherapy or combination therapy) for unresectable or metastatic solid tumors
having at least one measurable lesion according to RECIST v1.1
no history of other active malignancy within 3 years prior to study initiation (except curatively treated non-melanoma skin cancer, superficial bladder cancer, or intraepithelial carcinoma of the prostate, cervix, breast, or stomach)
no history of active autoimmune disease requiring systemic therapy
no concurrent condition requiring immunosuppressant or active infection
no contraindications to colonoscopy

### Example 3. Combination Therapy of FMT and Anti-PD-(L)1 Inhibitor

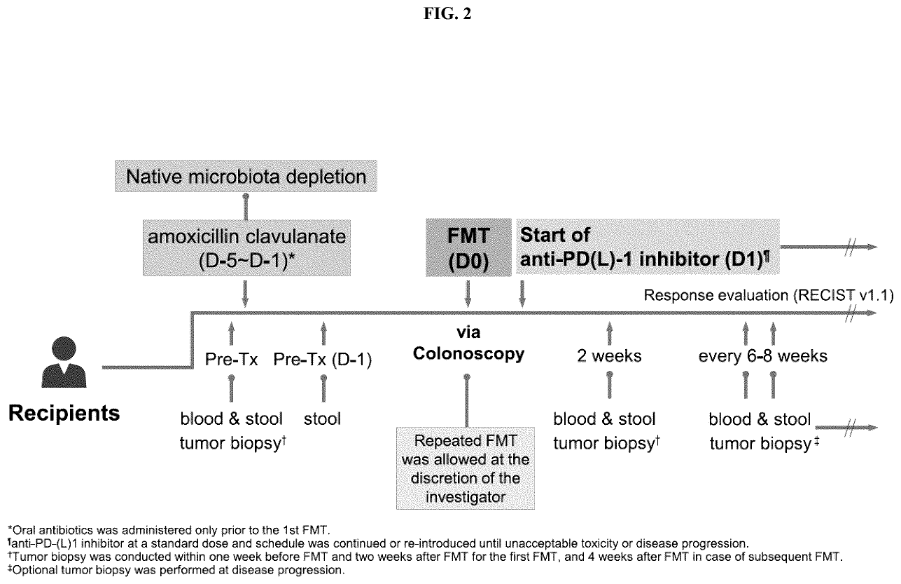

Combination therapy of FMT and an anti-PD-(L)1 inhibitor was performed on recipients satisfying the conditions described in Example 2, and this process is schematically shown in FIG. 2.

Specifically, a clinical trial for FMT and anti-PD-(L)1 inhibitor combination therapy was performed on patients (recipients) with unresectable or metastatic solid tumors whose cancer has progressed during anti-PD-(L)1 inhibitor treatment, and stool donors for FMT were patients with unresectable or metastatic solid tumors who had maintained complete response (CR) or partial response (PR) to anti-PD-(L)1 monotherapy for at least 6 months. To reduce the gut microbiome originally present in each recipient, an oral antibiotic (Augmentin®: amoxicillin clavulanate; 625 mg, three times a day) was administered for 5 days (from D-5 to D-1) before FMT, and the final antibiotic administration was completed at least 12 hours before FMT. FMT was administered via colonoscopy, and pre-colonoscopy bowel preparation was performed in accordance with the hospital's protocol. The thawed donor's stool fluid was administered into the recipients' intestines through the colonoscopy. On the day after FMT, the recipients received a standard dose of the anti-PD-(L)1 inhibitor, and such administration was continued until cancer progression or intolerable toxicity occurred. Additional FMT using feces of the same or different donor was permitted at the researcher's discretion, either before the tumor response assessment or in cases where the clinical responses were not clear. The tumor response assessment was conducted every 6 to 8 weeks through computed tomography according to RECIST v1.1.

3 mg/kg of nivolumab as an anti-PD-1 inhibitor was administered along with FMT via intravenous injection (IV) into all recipients participating in this study every two weeks. The median administration number of nivolumab administered to the 13 recipients was 5 cycles (one administration is considered one cycle), with a range of 1 to 27 cycles depending on the patient. Among the 13 recipients, 7 received at least two FMTs from the same or different donors; 4 received 2 FMTs; 2 received 3 FMTs; and 1 received 4 FMTs.

Amoxicillin/clavulanate, used as an oral antibiotic, were administered only before the first FMT. The anti-PD-(L)1 inhibitor was continued or reintroduced at the standard dose and schedule until unacceptable toxicity or disease progression.

Tumor biopsy was performed within 1 week before FMT, for the first FMT, within 2 weeks after FMT, and for subsequent FMTs, within 4 weeks after FMT. Selective tumor biopsy was performed during disease progression.

### Example 4. Clinical Characteristics

The clinical characteristics of the FMT recipients (N=13) of the present invention are shown in Table 1 below.

TABLE 1

| | N (%) |
|---|---|
| Age, years | 60 (38-76) |
| Sex | |
| Male | 10 (76.9%) |
| Female | 3 (23.1%) |
| ECOG performance status | |
| 0 | 1 (7.7%) |
| 1 | 2 (92.3%) |
| Types of cancer | |
| Gastrio adenocarcinoma (GC) | 4 (30.8%) |
| Esophageal SqCC (ESCC) | 5 (38.5%) |
| Hepatocellular carcinoma (HCC) | 4(30.8%) |
| Disease status | |
| Metastatic | 13 (100%) |
| Types of prior ICI | |
| Nivolumab monotherapy | 13 (100%) |
| No. of prior therapies | |
| for metastatic disease | |
| 2nd line | 1 (7.7%) |
| 3rd line | 7 (53.8%) |
| ≥4th line | 5 (38.5%) |
| Best response to prior | |
| ICI before FMT* | |
| CR | 0 (0%) |
| PR | 5 (38.5%) |
| SD | 2 (15.4%) |
| PD | 5 (46.2%) |
| Time from prior ICI dose to | 22 (16-98) |
| first study ICI dose (days) | |
| PD-L1 status | |
| Positive | 5 (38.5%) |
| Negative | 8 (61.5%) |
| Tumor mutation burden (muts/Mb) | 15.3 (10.9-28.1) |
| Microsatellite status, MSS | 13 (100%) |

Data are median (range) or number (%).
*Response was assessed using RECIST v1.1.
ICI = immune checkpoint inhibitor;
MSS = microsatellite stable.

In addition, the clinical characteristics of the FMT donors (N=6) are shown in Table 2 below.

TABLE 2

| | | | Cancer | | Response | DoR | TMB | MSI | PD-L1 | EBV |
|---|---|---|---|---|---|---|---|---|---|---|
| | Age | Sex | type | Anti-PD-1 | to anti-PD-1 | (months) | (/Mb)* | status | status | status |
| Donor #1 | 78 | Male | HCC | Pembrolizumab | CR | 58.7+ | 9.4 | MSS | TPS 0%, | NA |
| | | | | | | | | | CPS 0 | |
| Donor #2 | 66 | Male | GC | Nivolumab | CR | 72.7+ | 37.5 | MSS | TPS 7%, | negative |
| | | | | | | | | | CPS 7 | |
| Donor #3 | 63 | Male | HCC | Nivolumab | PR | 7.9+ | 15.6 | MSS | TPS 0%, | NA |
| | | | | | | | | | CPS 0 | |
| Donor #4 | 78 | Male | ESCC | Nivolumab | PR | 15.4+ | 10.9 | MSS | TPS 0%, | NA |
| | | | | | | | | | CPS 0 | |
| Donor #5 | 62 | Male | HCC | Nivolumab | CR | 31.5+ | NA | MSS | TPS 0%, | NA |
| | | | | | | | | | CPS 0 | |
| Donor #6 | 69 | Male | HCC | Nivolumab | CR | 25.7+ | 10.9 | MSS | TPS 0%, | NA |
| | | | | | | | | | CPS 0 | |

*In-house targeted NGS panel. Response was assessed using RECIST v1.1. DoR = duration of response; TMB = tumor mutation burden; MSI = microsatellite instability; MSS = microsatellite stable; TPS = tumor proportion score; CPS = combined positive score; ESV = Epstein-Barr virus; NA = not available.

In addition, side effects related to the combination treatment of FMT and the anti-PD-(L)1 inhibitor were confirmed in the FMT recipients. The results are shown in Table 3 below.

The side effects included skin rashes, skin pruritus, hypothyroidism, adrenal insufficiency, myalgia, fever, and gastritis.

TABLE 3

| | CTCAE v5.0, N (%) | | | |
|---|---|---|---|---|
| | Grade 1 | Grade 2 | Grade 3 | Grade 4 |
| Skin rash | 1 (7.7%) | 1 (7.7%) | 0 (0%) | 0 (0%) |
| Skin pruritus | 2 (15.4%) | 3 (23.1%) | 0 (0%) | — |
| Hypothyroidism | 0 (0%) | 2 (15.4%)* | 0 (0%) | 0 (0%) |
| Adrenal insufficiency | 0 (0%) | 1 (7.7%) | 0 (0%) | 0 (0%) |
| Myalgia | 1 (7.7%) | 0 (0%) | 0 (0%) | — |
| Fever | 1 (7.7%) | 0 (0%) | 0 (0%) | 0 (0%) |
| Gastritis | 0 (0%) | 0 (0%) | 1 (7.7%) | 0 (0%) |

*One case was subclinical hypothyroidism with a TSH level >10 mIU/L

### Experimental Example

### Experimental Example 1. Confirmation of Microbial Composition after FMT

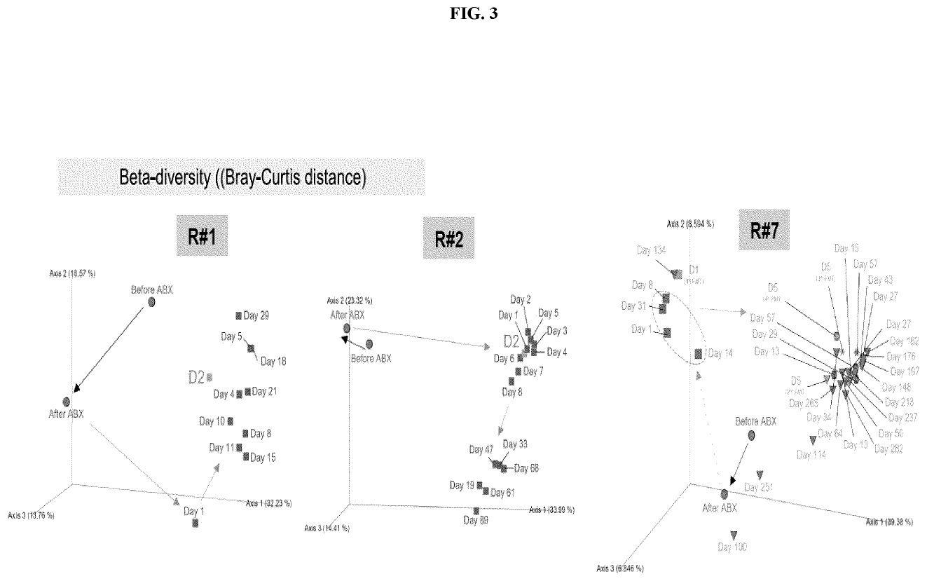

As a result of confirming the microbial composition in most recipients after FMT through beta-diversity (Bray-Curtis distance) analysis, the recipients underwent a significant change in microbial composition, and among these recipients, recipients R #1, R #2, and R #7, as shown in FIG. 3, exhibited microbial compositions that are very similar to those of their respective donors.

### Experimental Example 2. Confirmation of Clinical Efficacy

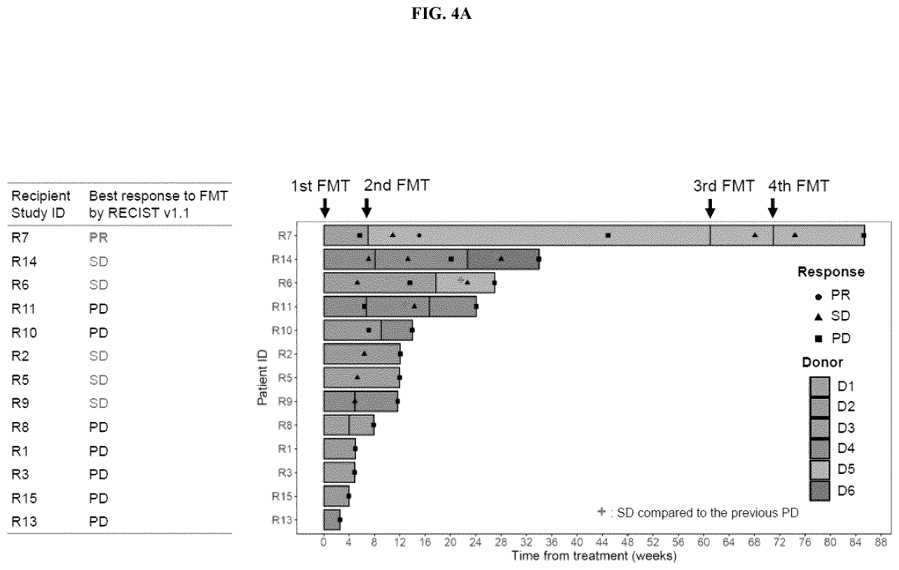

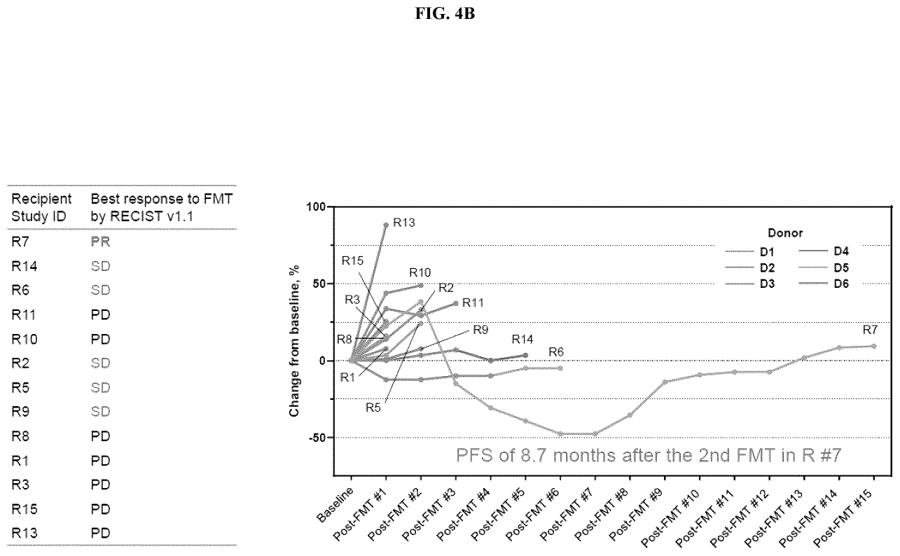

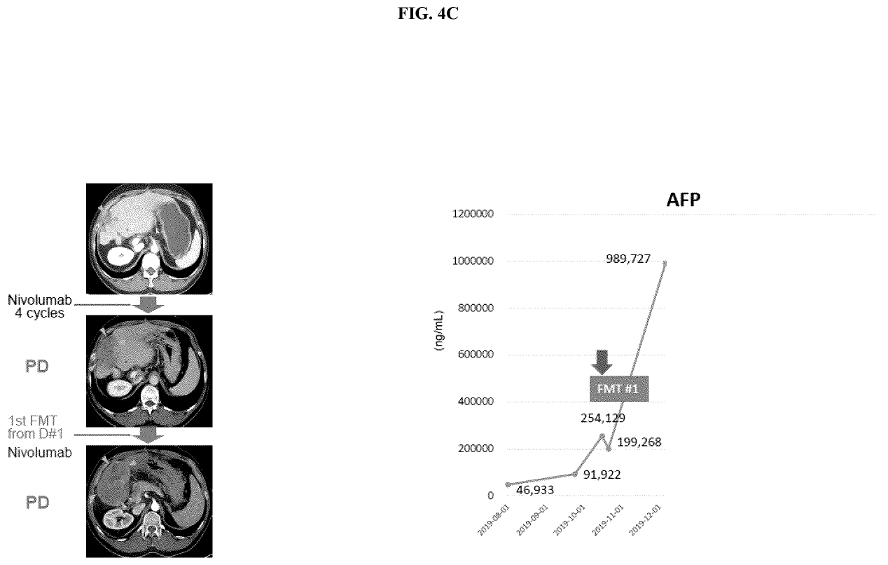

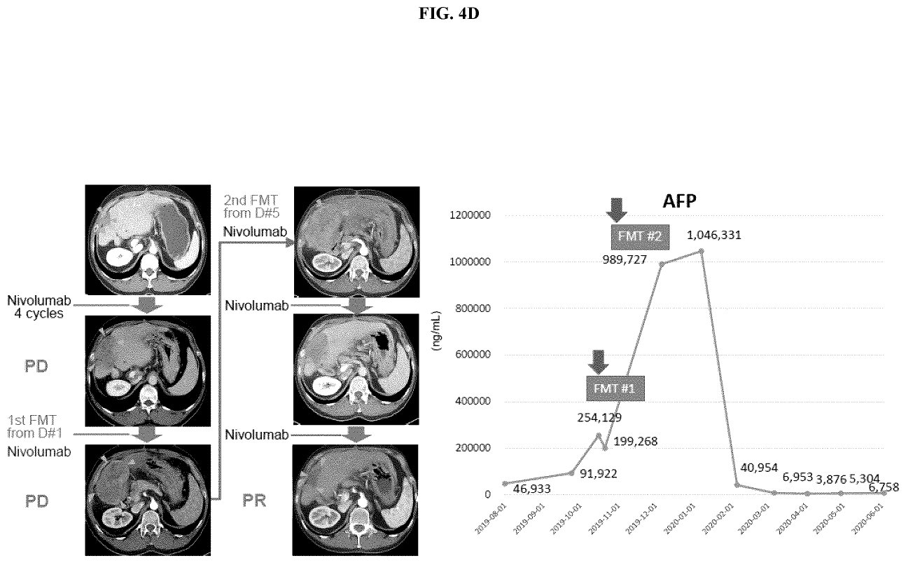

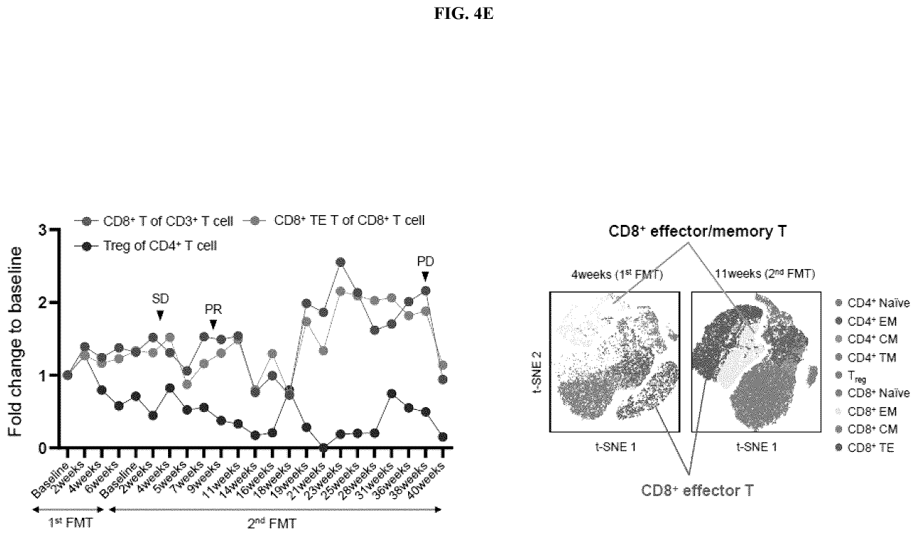

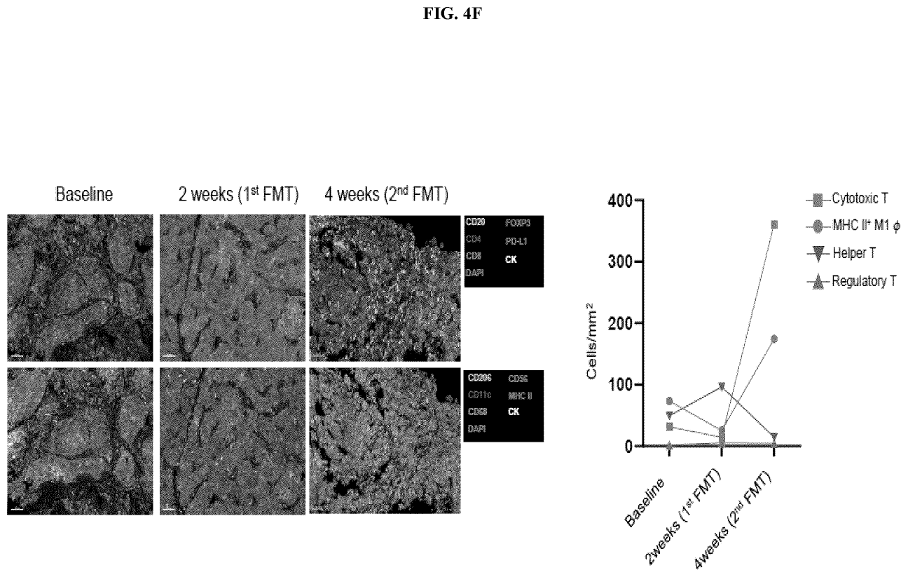

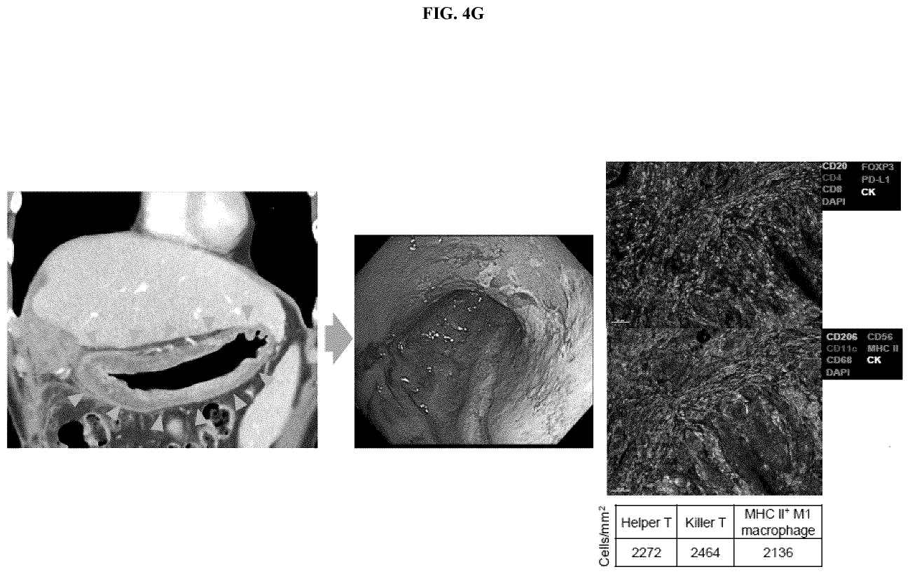

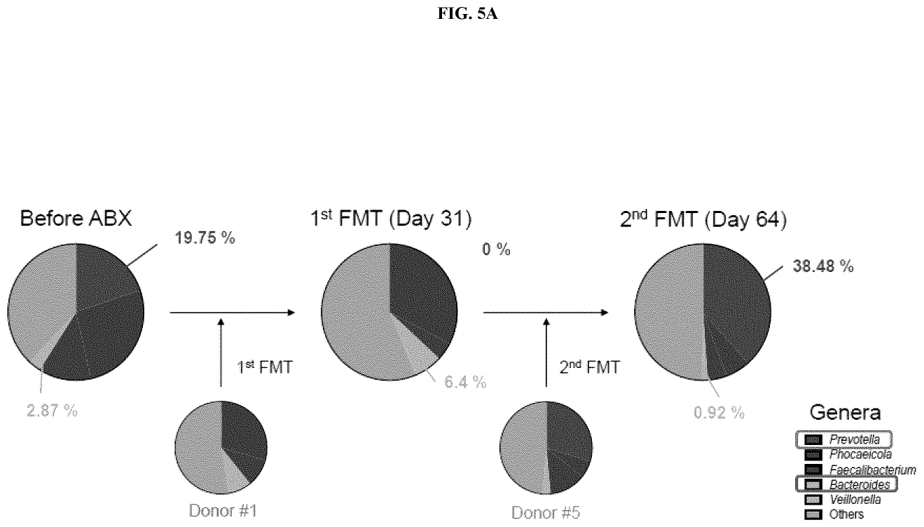

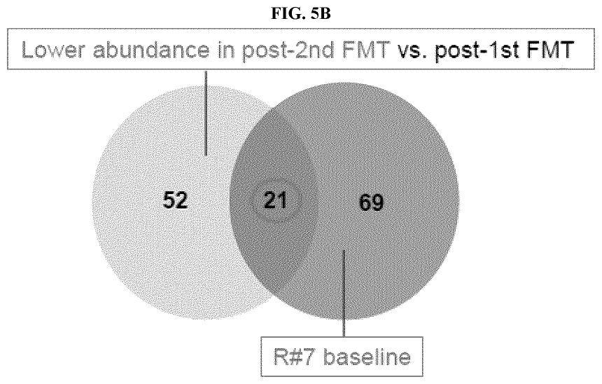

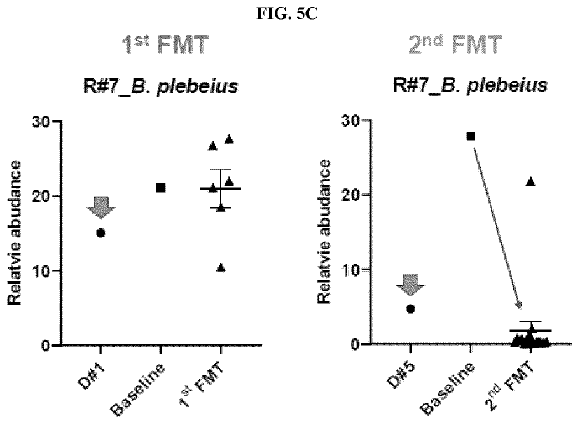

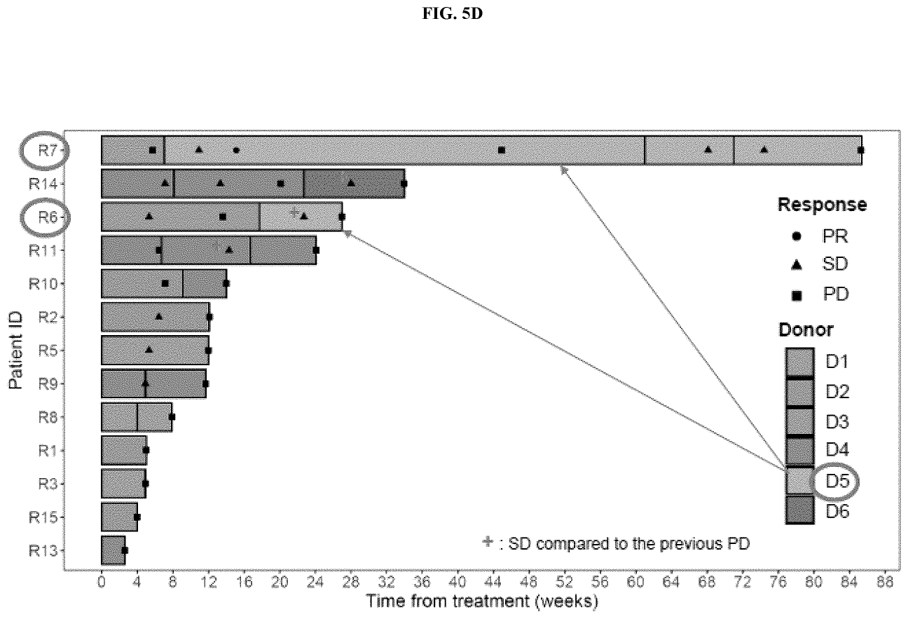

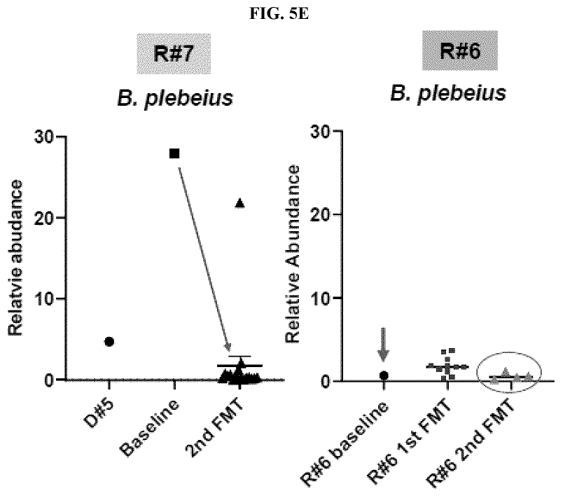

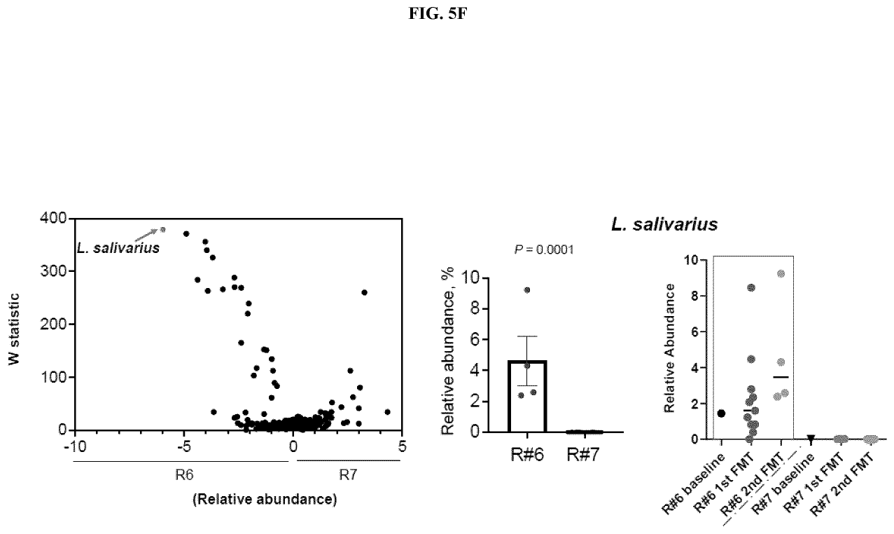

As a result of confirming clinical efficacy for the 13 recipients, as shown in FIGS. 4A and 4B, 5 of the recipients showed a stable disease (SD), 1 (R #7) achieved partial response (PR) after FMT, resulting in a disease control rate of 46.2% (6/13) and an objective response rate of 7.7%.

Therefore, various clinical responses to nivolumab administration were identified in the FMT recipient R #7 who achieved a PR.

### 2-1. Confirmation of α-Fetoprotein in Recipient R #7

As shown in FIG. 4C, in recipient R #7 (male, age 47, hepatocellular carcinoma) who showed progressive disease (PD) after 4 cycles of nivolumab administration, when a first FMT from donor D #1 was performed and nivolumab was administered, cancer progression continued, and one month after FMT, an increase in α-fetoprotein (AFP) was confirmed.

In addition, as shown in FIG. 4D, in recipient R #7, when the second FMT from donor D #5 was performed 7 weeks after the first FMT, and nivolumab was additionally administered twice at 2-week intervals, it was confirmed that a PR was exhibited, and three months after the second FMT, AFP significantly decreased.

Therefore, recipient R #7 had no enhanced therapeutic responsiveness to nivolumab after the first FMT, but an improved therapeutic responsiveness to nivolumab was observed after the second FMT.

### 2-2. Confirmation of Systemic Immune Activation in Recipient R #7

Mass cytometry was performed using cytometry by time of flight (CyTOF).

Specifically, PBMCs were obtained from patients using a Maxpar Direct Immune Profiling Assay kit (Fluidigm), and then the Fc receptors of immune cells were blocked using human TruStain FcX (BioLegend). Afterward, the cells were cultured with antibodies capable of detecting markers and Cell ID Intercalator-Ir (Fluidigm) for cell identification. Subsequently, changes in the immune cell system were confirmed through the expression of markers in each cell using a Helios mass cytometer (Fluidigm). This analysis evaluated systemic immune changes in peripheral blood from FMT recipient R #7.

As a result, as shown in FIG. 4E, compared to the baseline, it was confirmed that CD8⁺ T cells and CD8⁺ effector T cells increased after the second FMT, whereas regulatory T cells (Treg) decreased. In FIG. 4E, EM indicates effector memory, CM indicates central memory, TM indicates terminal memory, and TE indicates terminal effector.

### 2-3. Confirmation of Immune Activation in Tumor Microenvironment (TME) of Recipient R #7

Immune activation in TME in FMT recipient R #7 was confirmed through multiplex immunohistochemistry (IHC). Specifically, 4 μm-thick slices were cut from a formalin-fixed, paraffin-embedded (FFPE) block, and the slices were heated in a 60° C. dry oven for at least 1 hour. Afterward, multiplex immunofluorescence staining was performed using a Leica Bond Rx™ automated stainer (Leica Biosystems). Multiplex staining was performed through the following steps: antigen retrieval, blocking, antibody staining, tyramide signal amplification, and removal of bound antibodies for multiple antigens.

As a result, as shown in FIG. 4F, on week 4 after the second FMT, it was confirmed that significant increases in tumor-infiltrating cytotoxic T cells and MHC-II⁺ M1 macrophages were observed, and the number of Treg cells remained consistently low.

### 2-4. Confirmation of Immune-Related Gastritis in Recipient R #7

The result in which immune-related gastritis caused by immune activation following the second FMT occurred in FMT recipient R #7 was confirmed by upper endoscopy, histological examination, and clinical evaluation, and is shown in FIG. 4G.

### Experimental Example 3. Confirmation of Causative Bacteria Responsible for Therapeutic Effect of FMT

### 3-1. Confirmation of Changes in Genus-Level Abundance in Recipient R #7

Metagenomic analysis of patients' stool samples was performed using 16s rRNA sequencing, and microbial abundance was analyzed using QIIME2 and DADA2 tools. As a result of confirming the changes in genus-level microbial abundance in FMT recipient R #7, as shown in FIG. 5A, it was confirmed that, after the first FMT from donor D #1, the genus Bacteroides increased, the genus Prevotella decreased, and after the second FMT from donor D #5, the genus Bacteroides decreased and the genus Prevotella increased

### 3-2. Confirmation of Microorganisms with Decreased Abundance Following Second FMT in Recipient R #7

As a result of confirming microbial abundance after FMT in recipient R #7, as shown in FIG. 5B and Table 4 below, compared to the first FMT, 73 microorganisms showed decreased abundance following the second FMT, and 21 of these microorganisms were originally present in R #7, but were markedly reduced after the second FMT compared to the first FMT.

TABLE 4

| | | LefSe analysis | | Relative |
|---|---|---|---|---|
| | | (1st FMT > | | abundance |
| | | 2nd FMT) | | in R7 |
| No. | Name | LDA score | P-value | baseline |
| 1 | Bacteroides plebeius | 5.061387 | 0.001040 | 21.112934 |
| | DSM 17135 | | | |
| 2 | Bifidobacterium longum | 4.461033 | 0.001040 | 0.542587 |
| 3 | Bacteroides vulgatus | 4.262785 | 0.009087 | 5.239117 |
| 4 | Phascolarctobacterium | 4.244568 | 0.005770 | 0.012618 |
| | succinatutens | | | |
| 5 | g_Roseburia; _ | 4.066425 | 0.043962 | 0.446688 |
| 6 | g_Blautia; _ | 4.040936 | 0.025347 | 0.938801 |
| 7 | Bacteroides stercoris | 4.039197 | 0.003650 | 0.280126 |
| | ATCC 43183 | | | |
| 8 | Faecalibacterium longum | 4.034910 | 0.013906 | 8.522397 |
| 9 | Faecalibacillus intestinalis | 3.906048 | 0.000994 | 0.007571 |
| 10 | Collinsella aerofaciens | 3.663684 | 0.020855 | 0.540063 |
| 11 | Ligilactobacillus salivarius | 3.338701 | 0.006077 | 0.010095 |
| 12 | Bacteroides xylanisolvens XB1A | 3.281749 | 0.012513 | 0.065615 |
| 13 | Bacteroides thetaiotaomicron | 3.279202 | 0.037995 | 0.272555 |
| 14 | g_Bacteroides;_ | 3.224612 | 0.003799 | 0.315457 |
| 15 | Stenotrophomonas maltophilia | 3.157466 | 0.006077 | 0.015142 |
| 16 | Erysipelatoclostridium ramosum | 2.992714 | 0.000049 | 0.022713 |
| 17 | g_Pseudomonas; _ | 2.801149 | 0.006488 | 0.174132 |
| 18 | Parasutterella excrementihominis | 2.791501 | 0.000589 | 3.994953 |
| 19 | Chryseobacterium gambrini | 2.767977 | 0.001296 | 0.058044 |
| 20 | Hungatella hathewayi | 2.694359 | 0.003364 | 0.075710 |
| 21 | Moraxella osloensis | 2.643420 | 0.030871 | 0.035331 |

In addition, as confirmed in Experimental Example 2, recipient R #7 of the patients in the FMT cohort did not show an enhanced therapeutic responsiveness to an immune anticancer agent after the first FMT, but did show an improved therapeutic responsiveness to an immune anticancer agent after the second FMT. Therefore, as a result of analyzing strains that are abundant in the first FMT donor, compared to the second FMT donor, and significantly abundant after the first FMT compared to after the second FMT in patient R #7, as shown in FIG. 5C, the Bacteroides plebeius strain was selected.

In addition, as the B. plebeius strain is a strain originally present in patient R #7, it can be seen that it is a strain that suppresses the therapeutic effect of an immune anticancer agent.

### 3-3. Comparison of Responses in Recipients Receiving FMT from Donor D #5

Recipient R #6 patient of the patients in the FMT cohort received a second FMT from a donor (D #5) like patient R #7 did. As a result of confirming responses to the treatment with an immune anticancer agent in patients R #6 and R #7, as shown in FIG. 5D, partial response (PR) was sustained in patient R #7, whereas stable disease (SD) persisted only briefly in patient R #6, which indicates that, unlike patient R #7, patient R #6 did not have an enhanced therapeutic responsiveness to immune anticancer agent treatment.

In addition, as a result of comparing the abundance of B. plebeius in patients R #6 and R #7, as shown in FIG. 5E, B. plebeius abundance significantly decreased after the second FMT in patient R #7, whereas in patient R #6, B. plebeius abundance was low to begin with and did not increase after the first and second FMTs.

In addition, as a result of analyzing more strains in patient R #6 than patient R #7 after the second FMT using QIIME2 and Analysis of Composition of Microbiomes (ANCOM) (Mandal, S., Van Treuren, W., White, R. A., EggesbøM., Knight, R., and Peddada, S. D. (2015). Analysis of composition of microbiomes: a novel method for studying microbial composition. Microbial Ecology in Health and Disease 26, 27663.), as shown in FIG. 5F, the Lactobacillus salivarius strain was selected. The L. salivarius strain was originally present in patient R #6, and was maintained or increased even after FMT. Since this strain was also present during the first FMT in patient R #7, who did not show an enhanced therapeutic responsiveness to an immune anticancer agent, it can be seen that this strain suppresses the therapeutic effect of immune anticancer agents.

### Experimental Example 4. Confirmation of Immunosuppression Activity of B. plebeius and L. salivarius Strains

### 4-1. Confirmation of Inhibitory Effect on T Cell Proliferation

To confirm the immunosuppressive activity of the B. plebeius and L. salivarius strains, T cells and each strain culture fluid were co-cultured to confirm T cell proliferation inhibitory activity using flow cytometry.

First, peripheral blood mononuclear cells (PBMCs) were isolated from human blood using Ficoll, and then remaining red blood cells (RBCs) were removed using an RBC lysis buffer. Afterward, CD4 T cells and CD8 T cells were each isolated using a CD4/CD8 T cell isolation kit (MACS kit) and a LS column and stained with CFSE. Each type of cells were dispensed at 2×10⁵ cells/200 μL into a 96-well plate, and treated with the strain culture fluid. The 96-well plate was precoated with anti-CD3 and anti-CD28 antibodies, which can induce T cell proliferation, 24 hours in advance. The strain culture fluid was obtained after at least 12 hours of culture in an RPMI medium, and when treating T cells, 10% of the final T cell medium volume was treated. After 72-hour co-culture, T cells of each well were collected and CFSE fluorescence was detected using flow cytometry. When T cells proliferate extensively, the fluorescence intensity per individual T cell decreases. That is, decreased CFSE intensity or a shift of a peak to a lower value indicates that T cell proliferation has occurred. By using such an analysis method, the degree of T cell proliferation by each strain was analyzed.

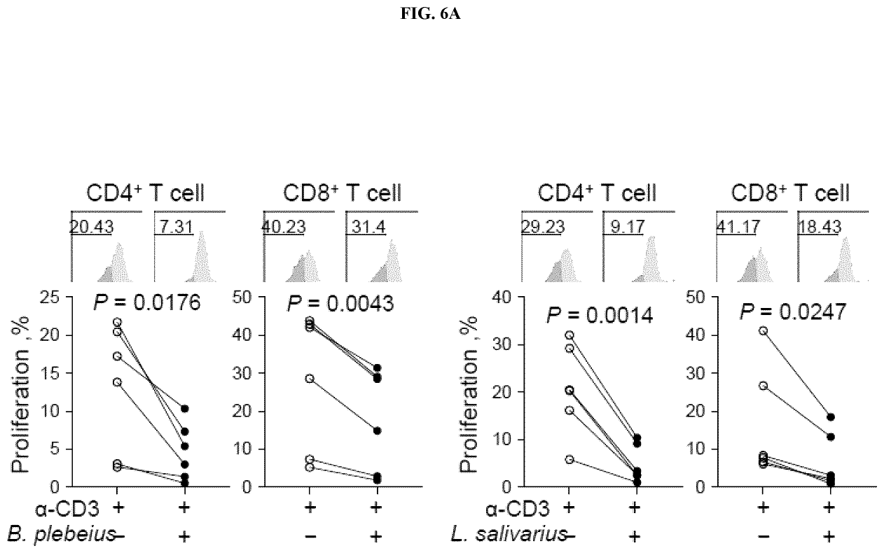

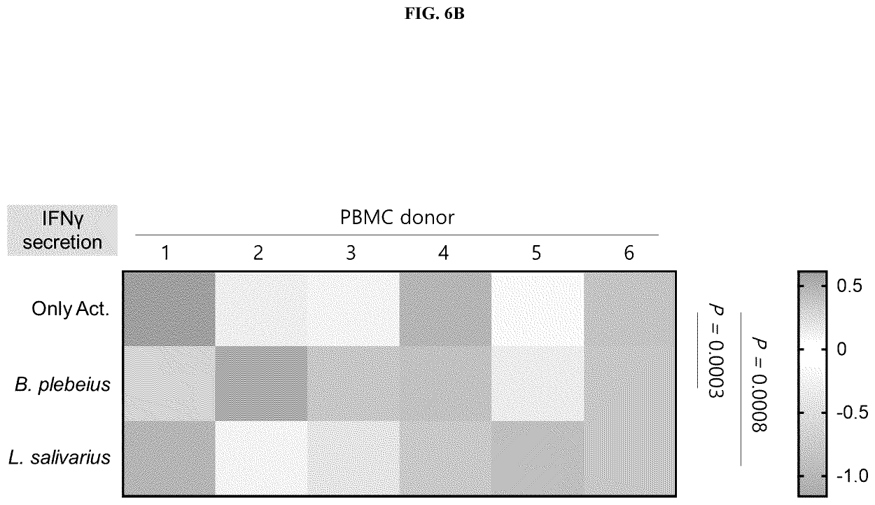

As a result, as shown in FIG. 6A, it was confirmed that when the B. plebeius and L. salivarius strain culture fluids were treated, it was confirmed that the proliferation of both CD4 T cells and CD8 T cells was inhibited.

### 4-2. Confirmation of Inhibitory Effect on Interferon-Gamma (IFN-γ) Secretion of T Cells

A co-culture fluid in which T cells and a strain culture fluid were co-cultured by the method of Experimental Example 4-1 was collected, and the concentration of IFN-γ secreted by the T cells was assessed using ELISA. Since the co-culture fluid is in a state in which IFN-γ is secreted as the T cells proliferate and activate, a capture antibody that is able to detect IFN-γ in the co-culture fluid was coated on a 96-well plate, treated with the co-culture fluid, and then cultured at room temperature for 1 hour to induce the binding of the capture antibody to IFN-γ. Afterward, the resulting plate was subjected to washing, staining with a second antibody, washing, and staining with an HRP reagent, followed by measuring the absorbance of each well using a microplate reader. The absorbance of each well was converted to the concentration of IFN-γ using reference wells (wells treated with known concentrations of IFN-γ), and the induced secretion levels of IFN-γ were compared and analyzed per bacterial strain. As a result, as shown in FIG. 6B, when treated with the B. plebeius and L. salivarius strain culture fluids, compared to the control, it was confirmed that the IFN-γ concentration was significantly reduced.

Therefore, it was confirmed from Experimental Examples 4-1 and 4-2 that the B. plebeius and L. salivarius strains can inhibit host immune activity.

### Experimental Example 5. Confirmation of Survival Rates According to Presence or Absence of B. plebeius and L. Salivarius Strains

Survival rates were analyzed according to the presence or absence of the B. plebeius and L. salivarius strains using an independent biomarker cohort. The analysis was performed on a total of 72 patients who had available baseline stool samples and had not received antibiotics within 4 weeks prior to the treatment with an immune checkpoint inhibitor (ICI). Their clinical information is shown in Table 5 below.

TABLE 5

| | N (%) |
|---|---|
| Age, years | 60 (37-83) |
| Sex | |
| Male | 59 (81.9%) |
| Female | 13 (18.1%) |
| Types of cancer | |
| Gastric adenocarcinoma | 27 (37.5%) |
| Esophageal SqCC | 23 (31.9%) |
| Hepatocellular carcinoma | 22 (30.6%) |
| Disease status | |
| Metastatic | 72 (100%) |
| Type of ICI | |
| Nivolumab | 72 (100%) |
| Line of ICIs for metastatic disease | |
| 2nd line | 6 (8.3%) |
| 3rd line | 47 (65.3%) |
| ≥4th line | 19 (26.4%) |
| No. of cycles of ICI administered | 8 (1-47) |
| Best response to ICI | |
| CR | 2 (2.8%) |
| PR | 8 (11.1%) |
| SD | 7 (9.7%) |
| NonCR/nonPD | 2 (2.8%) |
| PD | 53 (73.6) |

Data are median (range) or number (%)
*Response was assessed using RECIST v1.1.
ICI = immune checkpoint inhibitor.

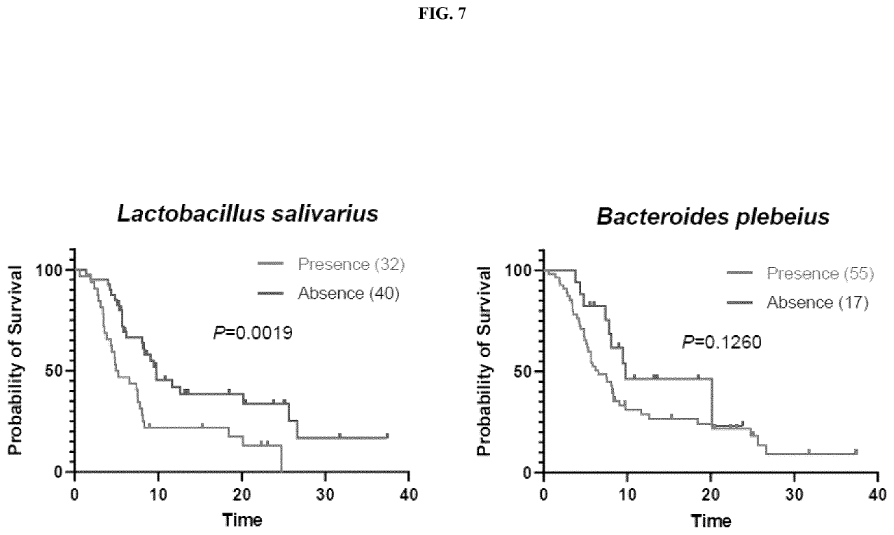

As a result of confirming the survival rates according to the presence or absence of the two strains in an independent biomarker cohort, as shown in FIG. 7, it was confirmed that the overall survival (OS) period was significantly longer in the patient group without the B. plebeius strain, and for L. salivarius, there was a significant difference in overall survival period depending on its presence or absence.

The foregoing description of the present invention is intended for illustrative purposes, and it will be understood by those skilled in the art that various modifications can be made thereto in other specific forms without departing from the technical spirit or essential features of the present invention. Therefore, the embodiments described above should be understood as illustrative in all respects and not restrictive.

### INDUSTRIAL APPLICABILITY

The present invention is industrially applicable, as it enables the prediction of therapeutic responsiveness to an immune anticancer agent or prognosis after immune anticancer agent treatment by detecting Lactobacillus salivarius and Bacteroides plebeius strains, and the strains are expected to be useful for therapeutic applications such as the treatment of autoimmune diseases through suppression of immune activity.

1. An analytical method for determining whether a subject receiving treatment with an anticancer agent has susceptibility or resistance to an immune anticancer agent, the method comprising:
detecting an intestinal microorganism comprising Lactobacillus salivarius and/or Bacteroides plebeius in a biological sample isolated from a subject.

2. The analytical method of claim 1, wherein the immune anticancer agent comprises an immune checkpoint inhibitor, an immune cell therapeutic agent, and/or an oncolytic viral therapeutic agent.

3. The analytical method of claim 2, wherein the immune checkpoint inhibitor is present and comprises a PD-1 inhibitor, a PD-L1 inhibitor, a CTLA4 inhibitor, a LAG3 inhibitor, a TIM3 inhibitor, a 4-1BB inhibitor, a B7-H4 inhibitor, a VISTA inhibitor, a KIR inhibitor, and/or a BTLA inhibitor.

4-10. (canceled)

11. The analytical method of claim 1, wherein the biological sample comprises blood, whole blood, plasma, stool, urine, tissue, cells, an organ, bone marrow, a fine-needle aspiration specimen, a fine-needle washout fluid, a core needle biopsy specimen, and/or saliva.

12. The analytical method of claim 1, further comprising:
predicting a low therapeutic responsiveness to an immune anticancer agent when the number of Lactobacillus salivarius or Bacteroides plebeius is higher than that of a control.

13. (canceled)

14. The analytical method of claim 1, further comprising:
predicting a poor prognosis of the subject after treatment with an immune anticancer agent when the number of Lactobacillus salivarius or Bacteroides plebeius is higher than that of a control.

15. A method for preventing or treating an autoimmune disease, the method comprising:
administering to a subject in need thereof a composition comprising Lactobacillus salivarius and/or Bacteroides plebeius, or a culture fluid thereof, as an active ingredient.

16. The method of claim 15, wherein the autoimmune disease is multiple sclerosis, inflammatory bowel disease, graft-versus-host disease, asthma, atopy, psoriasis, rheumatoid arthritis, and/or systemic lupus erythematosus.

17. The method of claim 15, wherein the composition suppresses immune activity.

18. The method of claim 17, wherein the composition inhibits T cell proliferation or reduces interferon-gamma (IFN-γ) secretion of T cells.

19-23. (canceled)

24. A method for treating cancer, comprising:
detecting a intestinal microorganisms comprising Lactobacillus salivarius and/or Bacteroides plebeius in a biological sample isolated from a subject;
predicting a high therapeutic responsiveness to an immune anticancer agent or a good prognosis of the subject after treatment with an immune anticancer agent when a number of Lactobacillus salivarius or Bacteroides plebeius is lower than or not detected compared to a subject having a low therapeutic responsiveness to an immune anticancer agent or a poor prognosis after treatment with an immune anticancer agent; and
treating the subject predicted to have high therapeutic responsiveness to an immune anticancer agent or good prognosis after treatment with an immune anticancer agent with the immune anticancer agent.

25. The method of claim 24, wherein the immune anticancer agent comprises an immune checkpoint inhibitor, an immune cell therapeutic agent, and/or an oncolytic viral therapeutic agent.

26. The method of claim 24, wherein the cancer is colorectal cancer, rectal cancer, colon cancer, thyroid cancer, oral cancer, pharyngeal cancer, laryngeal cancer, cervical cancer, brain cancer, glioblastoma, lung cancer, ovarian cancer, bladder cancer, kidney cancer, liver cancer, pancreatic cancer, prostate cancer, skin cancer, tongue cancer, breast cancer, uterine cancer, stomach cancer, bone cancer, lymphoma, blood cancer, epithelial squamous cell cancer, adenocarcinoma of the lung, peritoneal cancer, skin cancer, cutaneous melanoma, ocular melanoma, anal cancer, esophageal cancer, small intestine cancer, endocrine cancer, parathyroid cancer, adrenal cancer, soft tissue sarcoma, urethral cancer, astrocytoma, endometrial cancer, salivary gland cancer, vulvar cancer, and/or head and neck cancer.
